(19) United States
(12) Patent Application Publication
(10) Pub. No.: US 2012/0196760 A1
(43) Pub. Date: Aug. 2, 2012

(54) METHODS AND COMPOSITIONS FOR IDENTIFYING MODULATORS OF ANTI-TETHERIN ACTIVITY TO INHIBIT PROPAGATION OF VIRUSES

(75) Inventor: Paula Cannon, South Pasadena, CA (US)

(73) Assignee: CHILDRENS HOSPITAL LOS ANGELES, Los Angeles, CA (US)

(21) Appl. No.: 13/062,401

(22) PCT Filed: Sep. 4, 2009
(86) PCT No.: PCT/US09/56068

§ 371 Date: Mar. 4, 2011

Related U.S. Application Data
(60) Provisional application No. 61/191,151, filed on Sep. 4, 2008.
(60) Provisional application No. 61/196,291, filed on Oct. 16, 2008.

(51) Int. Cl. C40B 30/04; C12N 9/00; C12N 5/10; C12Q 1/68; C12N 1/21; C07K 19/00; C12N 15/62

(52) U.S. Cl. 506/9; 530/350; 435/183; 536/23.4; 435/6.1; 435/252.3; 435/367

(57) ABSTRACT

The present invention provides protein constructs for detection of interaction between Tetherin and ant-Tetherin molecules. The constructs include a Tetherin sequence or a portion of a Tetherin sequence that is sufficient for cell-surface localization, and a sequence providing a detectable signal. The invention further provides methods of using such constructs to screen for substances that modulate the Tetherin-inhibiting properties of anti-Tetherins. Methods of screening for anti-viral agents, including anti-HIV agents, are provided.

### CROSS-REFERENCE TO RELATED APPLICATIONS

The present application relies on and claims the benefit of the filing date of U.S. provisional patent application No. 61/191,151, filed 4 Sep. 2008, and U.S. provisional patent application No. 61/196,291, filed 16 Oct. 2008, the entire disclosures of both of which are hereby incorporated herein by reference.

### STATEMENT OF GOVERNMENT INTEREST

This invention was made partially with U.S. Government support from the United States National Institutes of Health under Grant Number 5R01 AI068546. The U.S. Government has certain rights in the invention.

### BACKGROUND OF THE INVENTION

1. Field of the Invention

The present invention relates to the fields of medicine and clinical diagnostics. More specifically, the invention relates to proteins, compositions, and methods that are useful in identifying modulators of anti-Tetherin activity in viruses, including human immunodeficiency virus (HIV) and other lentiviruses.

2. Description of Related Art

Certain human cells posses an activity that inhibits the release of retroviruses and other enveloped viruses from those cells. The activity is linked to molecules that tether the viral particles to the cells, and those molecules have therefore been termed “Tetherins”. The human protein BST-2/CD317/HM1.24/Tetherin has recently been identified as a cellular factor that tethers newly budded HIV particles at the surface of a cell, and thereby reduces the yield of infectious virions (Neil et al., 2008, Nature 451:425-430; Van Damme et al., 2008, Cell Host. Microbe 3:245-252). Its mechanism of action is presently unknown, but the fact that Tetherin exists as a homodimer, with each monomer anchored in the plasma membrane through both a membrane-spanning sequence and a GPI anchor, has led to the suggestion that the protein could physically link viral and cellular membranes, preventing viral particle release from infected cells. Tetherin also restricts the release of other enveloped viruses, including other lentiviruses, retroviruses, filoviruses, herpesviruses, and arenaviruses, suggesting that it may be part of an innate cellular defense against enveloped viruses.

HIV codes for two distinct proteins that counteract the action of Tetherin, the HIV-1 Vpu protein and the HIV-2 Env protein (Anti-Tetherins) (Strebel et al., 1988, Science 241:1221-1223; Bour et al., 1996, J. Virol. 70:8285-8300; Noble et al., 2005, J. Virol. 79:3627-38). In addition, the Kaposi's sarcoma-associated herpesvirus (KSHV), which can be a significant cause of pathology in HIV-infected individuals, also targets Tetherin through the action of its K5 protein. There thus appears to be an evolutionarily developed response by viruses to overcome the Tetherin-directed cellular response to viral infection.

Very few drugs or viral inhibitors are known that act at late stages of the HIV life-cycle, such as at virus release. In part, this reflects the fact that these stages are difficult targets to analyze in standard high throughput screens (HTS). Typically such studies have used the secretion of virus-like particles (VLPs) into cell culture supernatants as the assay endpoint, to be measured after concentration and quantitation using enzyme assays (e.g., reverse transcriptase activity), by measurement of HIV antigens, or through the inclusion of covalently linked enzymatic reporters in the VLPs (e.g., alkaline phosphatase or β-lactamase). These assays are somewhat cumbersome, requiring harvesting and concentration of supernatants, and this significantly limits their application to HTS formats.

In view of the tremendous medical, economic, and societal impact of viral infections, including HIV infections, in humans, efforts continue to be made to develop bioactive substances (e.g., drugs) to interrupt viral replication and spread. The presence of proteins with anti-Tetherin activity in human viral pathogens suggests that the ability of certain viruses to inhibit Tetherins is important in the propagation of those viruses. The interaction between Tetherin and anti-Tetherin molecules might also be a potent target point for drugs to regulate and control viral activity. For example, a drug or therapy that is capable of modulating the ability of an Anti-Tetherin (for example, HIV-1 Vpu, HIV-2 Env protein, KSHV K5, and/or any other Anti-Tetherin) to inhibit the activity of Tetherin would be a valuable asset in the treatment of viral infection.

### SUMMARY OF THE INVENTION

The present invention provides engineered proteins and nucleic acids encoding them for use in screening for bioactive agents that reduce, inhibit, abolish, or otherwise modulate the ability of an anti-Tetherin to inhibit the activity of a Tetherin. The invention further provides recombinant cells for production of such engineered proteins, as well as for screening for such bioactive agents. The present invention further provides methods of screening for such bioactive agents, as well as kits for performing the screening methods. The invention provides for the use of Tetherin reporter constructs in determining the cellular localization of the constructs, and in particular determining whether the construct is localized to the cellular membrane. In embodiments, the constructs are also used to determine if the constructs are localized in the cellular cytoplasm. The invention also provides for use of nucleic acids encoding reporter constructs for production of the constructs. The invention further provides for use of host cells for production of the constructs and nucleic acids of the invention, for determining the cellular localization (e.g., cell surface localization) of the constructs, and for assays for modulators of anti-Tetherin activity of anti-Tetherins. In addition, the invention provides for use of kits to perform assays for determination of cellular localization of reporter constructs, and for identification of modulators of anti-Tetherin activity of anti-Tetherins.

The present invention provides surrogate Tetherin-based reporters that recapitulate the interaction of natural Tetherins and anti-Tetherins, and additionally allow for detection of the cellular localization of the reporters. The reporters form the basis of high-throughput screens for inhibitors that block the anti-Tetherin activity of proteins. In general, the engineered reporter molecules comprise a Tetherin or a portion of a Tetherin that is sufficient for both cell-surface membrane localization and anti-Tetherin interaction. The engineered reporters further comprise a detectable portion, such as a fluorescent moiety or an enzymatic moiety, that either intrinsically produces a detectable signal or can be a member or component of a system that produces a detectable signal. In exemplary embodiments, the reporter is a fusion protein that comprises the N-terminal 50 residues of human Tetherin fused to a detectable moiety.

The Tetherin-based reporters are encoded by nucleic acids of the invention. In general, the nucleic acids of the invention comprise engineered sequences that encode a Tetherin-derived sequence that is sufficient for both cell-surface membrane localization and anti-Tetherin interaction, which is fused in-frame to a coding sequence for a detectable moiety, such as an intrinsically fluorescent protein or an enzyme. The nucleic acids of the invention can comprise the coding sequence alone, but are more preferably provided as a cassette or other more sophisticated construct, which can include nucleic acid sequences for expression of the coding sequence, replication of the construct, and/or maintenance of the coding sequence in a host cell either transiently or stably. As such, the nucleic acid of the invention can be a vector, such as a plasmid, phagemid, or virus.

In one aspect of the invention, recombinant or host cells are provided. In some embodiments, the recombinant cells comprise a Tetherin-based reporter protein. In some embodiments, the recombinant cells comprise a nucleic acid encoding a Tetherin-based reporter protein. In some embodiments, the recombinant cell comprises both. The type of cell is not particularly limited, and thus can be prokaryotic or eukaryotic. Because the screening methods of the invention are preferably performed for the identification of bioactive agents relating to human disease, preferably the cells are human cells. However, it is to be recognized that other cells, including cells of other mammals, for example primates, can be used advantageously to both study the viral replication process and to identify bioactive agents that might have bioactivity in humans.

The invention further provides methods of screening for bioactive agents that modulate the activity of an anti-Tetherin on a Tetherin. While any type of modulation is encompassed by the invention, in preferred embodiments, the bioactive agent inhibits, abolishes, or otherwise reduces the ability of an anti-Tetherin to interact with a Tetherin. In general, the method of screening according to the invention comprises using a recombinant cell that comprises a Tetherin reporter construct of the invention and an anti-Tetherin to determine the cellular location of the Tetherin reporter construct. More specifically, because the Tetherin reporter constructs of the invention (1) are capable of properly locating to the cellular membrane, (2) are capable of being blocked from properly locating to the cellular membrane by anti-Tetherins, and (3) are capable of producing a detectable signal, the reporter constructs can be used in conjunction with anti-Tetherins to assay for interaction of the construct with the anti-Tetherin. As such, the presence of the construct within the cell (as determined by the location of the detectable signal) indicates whether interaction between the construct and the anti-Tetherin has occurred. In general, substantial detectable signal at the cell surface indicates that the anti-Tetherin did not interact with the Tetherin-derived reporter, whereas substantial detectable signal within the cell cytoplasm or reduction in overall Tetherin signal indicates an interaction between the anti-Tetherin and the construct. According to the method of the invention, simple optical detection of the location of the detectable signal allows a determination of the interaction of the anti-Tetherin with the Tetherin construct, indicating the effect of the bioactive agent on the interaction. Due to its relative ease of use, the method of the invention is well suited for HTS assays.

In yet another aspect, the invention provides kits for practicing the method of the invention. In general, the kits of the invention comprise a nucleic acid of the invention. Preferably, the kits further comprise a cell that expresses one or more anti-Tetherin molecules. In embodiments, the kits comprise host cells that express both a Tetherin-derived reporter and an anti-Tetherin. Of course, the kits of the invention can also include other reagents and supplies for practicing the method of the invention. While not limited to such a configuration, typically, the components of the kit are separately provided in independent containers, and the containers are packaged in combination to be provided as a kit.

### BRIEF DESCRIPTION OF THE DRAWINGS

The accompanying drawings, which are incorporated in and constitute a part of this specification, illustrate embodiments of the invention and provide experimental support for embodiments of the invention, and together with the written description, serve to explain certain principles of the invention.

FIG. 1 shows a sequence alignment of selected Tetherins from primate species. The approximate transmembrane region is indicated by asterisks.

FIG. 2, Panels A and B, show Western blots of HIV-1 Gag-Pol virus-like particles (VLPs) released from cells in the absence (lane 1) or presence of anti-Tetherins HIV-1 Vpu, HIV-2 Env, and KSHV K5, and the lack of effect of HIV-1 Env, which is not an anti-Tetherin and therefore serves as a negative control. Panel A shows the effects of the anti-Tetherins on VLP release from human HeLa cells, which naturally express Tetherin. Panel B shows the effects in simian Cos-7 cells, which do not express Tetherin, both in the absence (left) and presence (right) of human Tetherin.

FIG. 3, Panel A, shows a confocal micrograph of HeLa cells that naturally express endogenous Tetherin, with Tetherin detected using a specific antibody.

FIG. 3, Panel B, shows a cartoon representation of a Tetherin reporter construct, in which full-length human Tetherin is fused at its N-terminus to enhanced Green Fluorescent Protein (eGFP).

FIG. 3, Panel C, shows a confocal micrograph of HeLa cells expressing eGFP-Tetherin.

FIG. 3, Panel D, shows a confocal micrograph of HeLa cells co-expressing eGFP-Tetherin and Vpu, where the cellular distribution of eGFP-Tetherin is altered, as it is removed from the cell surface and concentrated in an intracellular peri-nuclear location that overlaps with Vpu (data not shown).

FIG. 3, Panel E, shows a bar graph indicating the cellular location of eGFP-Tetherin when expressed alone or in combination with Vpu or HIV-2 Env. “% cells” indicates the % of cells expressing a cell surface rim of eGFP-Tetherin, which is strongly reduced by expression of the anti-Tetherin factors Vpu or HIV-2 Env.

FIG. 4, Panel A, shows a cartoon representation of a Tetherin-derived reporter construct, mTG (SEQ ID NO:10), according to an embodiment of the invention.

FIG. 4, Panel B, shows a confocal micrograph of HeLa cells expressing the mTG construct. Similar to the situation with the full-length Tetherin in eGFP-Tetherin, mTG is found at the cell surface.

FIG. 4, Panel C, shows a confocal micrograph of HeLa cells co-expressing the mTG construct and HIV-1 Vpu. Similar to the situation with the full-length Tetherin in eGFP-Tetherin, mTG is removed from the cell surface by Vpu.

FIG. 4, Panel D, shows a graph indicating the relative amounts of mTG located on the cell surface of HeLa cells when expressed alone or in combination with HIV-1 Vpu. mTG levels were detected by staining the cells with an antibody to GFP and FACS analysis.

FIG. 5, Panel A, shows FACS analysis of cell-surface expression of mTG detected by staining the cells with an antibody to GFP and FACS analysis. The cells beyond the gate (line) have detectable eGFP on their cell surface.

FIG. 5, Panel B, shows FACS analysis of cell-surface expression of mTG when co-expressed with HIV-Vpu detected by staining the cells with an antibody to GFP and FACS analysis. Vpu co-transfection results in reduction in number of cells with eGFP on their cell surface.

FIG. 5, Panel C, shows a bar graph representing the data presented in FIG. 5, Panels A and B. Mock cells are HeLa cells stained with the anti-GFP antibody; eGFP cells are transfected with an expression plasmid for eGFP alone, which is an intracellular protein and so gives only background staining in this FACS assay.

FIG. 6 shows a bar graph representing the data presented in FIG. 5C, and additionally data relating to co-expression of mTG and KSH K5.

FIG. 7, Panels A and B, show confocal micrographs of expression of a Tetherin reporter construct containing a luciferase reporter, alone or co-expressed with HIV-1 Vpu. The luciferase moiety was detected using a specific antibody.

FIG. 8, Panels A and B, show confocal micrographs of expression of a Tetherin reporter construct containing a HaloTag™ (Promega, Madison, Wis.) reporter, alone or co-expressed with HIV-1 Vpu. HaloTag™ was detected using a HaloTag ligand.

FIG. 9, Panel A, depicts in cartoon form, various Tetherin reporter constructs in which the transmembrane domain (TMD), the cytoplasmic tail (CT), or both are replaced with the corresponding sequences from human transferrin receptor. Constructs also have eGFP labels, as indicated.

FIG. 9, Panel B, shows Western blot results and a corresponding bar graph of % VLP release, indicating the production of Virus-Like-Particles (VLPs) from cells expressing the constructs of Panel A in the presence or absence of various anti-Tetherins. Vpu (indicated as Vphu), HIV-2 Env, and the Ebola glycoprotein (GP) (solid arrows) indicate productive Tetherin/anti-Tetherin interactions that lead to increased release of HIV-1 VLPs compared to the levels with the Tetherin derivative alone; dashed arrows indicate non-functional interactions that do not stimulate VLP release. The data show that Vpu interacts with the TMD of Tetherin.

### DETAILED DESCRIPTION OF VARIOUS

### Embodiments of the Invention

Reference will now be made in detail to various exemplary embodiments of the invention. It is to be understood that the following detailed description of embodiments is provided to give the reader a better understanding of certain embodiments and features of the invention, and is not to be considered as a limitation on the scope or content of the invention, as broadly disclosed herein.

It has previously been reported that human (HeLa) cells restrict the release of HIV-1 virus-like particles (VLPs), while simian (Cos-7) cells do not. The basis for this restriction has recently been identified as the human BST-2/CD317/HM1.24/Tetherin. Both HIV-1 Vpu and the HIV-2 Env can counteract this restriction and thereby increase the level of VLPs released from HeLa cells. Likewise, the KSHV K5 protein shares this activity. It has also been found that adding human Tetherin to simian Cos-7 cells profoundly restricts VLP release, and that this restriction can be counteracted by both Vpu and HIV-2 Env. It thus is apparent that interaction between Tetherin and anti-Tetherin molecules play an important role in the viral replication and infection cycle. The present inventor has used this interaction to develop reporter constructs, cells, and assays for identification of substances that can interfere with the interaction between Tetherin and anti-Tetherin molecules. Such substances can represent drugs or lead compounds having anti-viral activity.

Viral Anti-Tetherins, such as HIV-1 Vpu and KSHV K5, are important drug targets. HIV-1 Vpu protein is essential for HIV-1 pathogenicity. It is currently an underdeveloped target for drugs, largely because there has been no simple assay to detect Vpu activity that could be amenable to high throughput screening (HTS). A challenge in doing HTS for inhibitors of Vpu-Tetherin interaction is that such an assay using currently available technology would rely on measuring budding of viruses or virus-like particles (VLPs) into a tissue culture supernatant. Such assays are currently done, for example, by harvesting VLPs from supernatants by ultracentrifugation, with subsequent detection of the VLPs by Western blotting, immunprecipitation, ELISAs or enzymatic activity (e.g., reverse transcriptase (RT) assays). The assays are labor intensive and not suitable for HTS.

The present invention overcomes the drawbacks inherent in screening methods based on currently available technology by providing protein constructs and assays that allow for rapid and large-scale identification of inhibition of Tetherin localization at the cellular membrane by anti-Tetherins. The constructs utilize a detectable reporter linked to Tetherin, or a portion of Tetherin sufficient for cell-surface localization and inhibition of localization by anti-Tetherins. The reporter constructs behave essentially identically to full-length, wild-type Tetherin with respect to cellular localization and inhibition by anti-Tetherins, yet at the same time include a detectable moiety that can be used to easily identify the cellular location of the reporter construct. The constructs and assays of the invention mimic the Tetherin-anti-Tetherin interaction and thus can be used to detect disruption of the interaction by drugs. The constructs and assays have broad application, and thus can be applied to other virus proteins, for example, to screen for inhibitors of the K5-Tetherin interaction, and any other viral Anti-Tetherin factors.

The methods (also referred to herein as “processes” or “assays”) of the current invention provide various advantages, some or all of which may be realized in one or more of the embodiments of the current invention. For example, the assays do not depend on quantitation of released VLPs and so do not require centrifugation of supernatant followed by immunological or enzymatic detection, steps that are not suitable for HTS. In addition, the methods of the invention are shown herein to recapitulate the key findings that Vpu and K5 remove Tetherin from the cell surface, thus showing that the assay is robust and has wide applicability. Furthermore, the assays separate the anti-Tetherin interaction with Tetherin from all other aspects of viral budding that could also be inhibited by drugs and lead to reduced VLP production (including non-specific effects on cell viability, global protein production etc.), and are therefore highly specific for the anti-Tetherin-Tetherin interaction. Such specificity reduces the number of false positives in HTS and greatly speeds the identification of specific inhibitors. Another advantage is that the assay readout is a positive effect, i.e., the expression of Tetherin at the cell surface. This is therefore likely to rule out non-specific (e.g., toxic) effects that could interfere with anti-Tetherin expression or activity, since such effects would also likely affect the expression or activity of the Tetherin, and thus would not be scored in the assay.

In a first general aspect of the invention, fusion constructs (also referred to herein as Tetherin reporters or similar terms) are provided. The fusion constructs comprise a Tetherin or a portion of a Tetherin that is sufficient to localize to a cell-surface membrane and to allow for inhibition of localization by an anti-Tetherin. In preferred embodiments, the Tetherin region of the construct comprises less than the full-length sequence of a wild-type, naturally occurring Tetherin. The human Tetherin protein is known and publicly available (e.g., GenBank), as are Tetherin proteins from other species (including various primates, pig, and rodents). The invention encompasses all Tetherin proteins from all species. Those of skill in the art are free to select the Tetherin protein of interest for any particular purpose, without departing from the scope of the invention.

In some embodiments, the fusion construct comprises less than the full amino acid sequence of a Tetherin protein. In such embodiments, the construct comprises a sequence derived from a Tetherin protein that includes a sufficient amino acid sequence to provide both cell-surface membrane localization and inhibition by one or more anti-Tetherins. For example, in exemplary embodiments discussed in detail below, an approximately 50 amino acid sequence at the N-terminus of human Tetherin (SEQ ID NO:11) is sufficient to provide these functions, and thus is used as the Tetherin portion of reporter constructs. Data disclosed herein shows that this 50 amino acid residue portion of human Tetherin fully recapitulates the cellular localization activity of Tetherin and the inhibition of Tetherin activity by anti-Tetherins. As such, portions of Tetherin comprising this 50 residue region can be used to create the reporter constructs of the invention.

It is to be recognized that the Tetherin sequence and partial sequence derived from it are not specifically limited to the precise sequence of the human Tetherin or a specific Tetherin from any other species. The present invention provides Tetherin-derived fusion constructs that recapitulate natural Tetherin activity with regard to cellular localization and inhibition by anti-Tetherins. As detailed below, these functions are provided by a transmembrane domain of Tetherin, preferably in conjunction with a cytoplasmic tail, features that are shared among Tetherins. While the sequence providing the transmembrane domain and, preferably cytoplasmic tail, has been determined to be important for the functions discussed herein, it is to be understood that certain variations in the sequence can be tolerated without completely abolishing the desired activity(ies). With regard to the transmembrane domain, those of skill in the art are fully aware that transmembrane domains (also referred to in the art as membrane-spanning regions) share certain physical characteristics. For example, they typically are defined by lengths of about 20 residues, are generally comprised of hydrophobic or non-polar residues, and generally do not include residues that cause inflexible bends or turns (e.g., typically do not comprise proline). Thus, those of skill in the art will recognize that certain conservative amino acid substitutions, deletions, or additions can be made to the specific sequence of the human Tetherin without abolishing its activity. Substitutions that may be made include, but are not limited to substitutions of one or more of the following amino acids with others of the group: alanine, cysteine, glycine, isoleucine, leucine, methionine, phenylalanine, proline, tryptophan, and valine. Non-limiting examples of residues that may be varied while still retaining activity include the following residues of the human Tetherin and the corresponding residues from other Tetherins: G25, I26, I28, L29, V30, I33, I34, I36, P40, I43, F44, and T45.

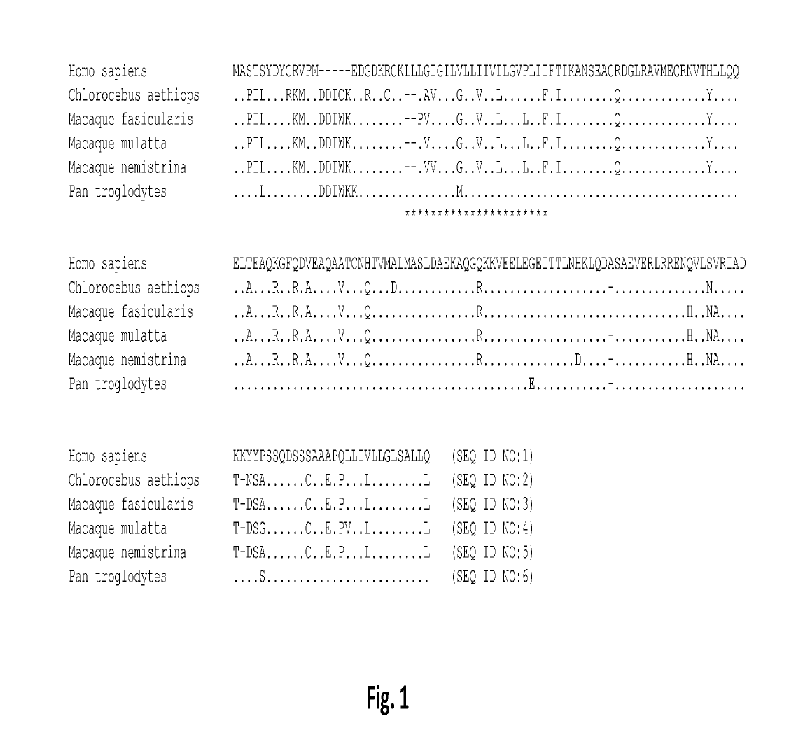

As shown in FIG. 1, there is extremely high sequence identity among the Tetherin proteins from primates. Those of skill in the art can use the information provided in FIG. 1 to make any number of Tetherin reporter constructs, recognizing that conserved residues should generally be maintained whereas variant residues can be altered. The information provided by this document shows that the portions of Tetherin C-terminal to the transmembrane domain can be eliminated while still retaining the desired activity. Thus, Tetherin reporter constructs can comprise any portion, or no portion at all, of a Tetherin protein that contains sequence C-terminal to the transmembrane domain.

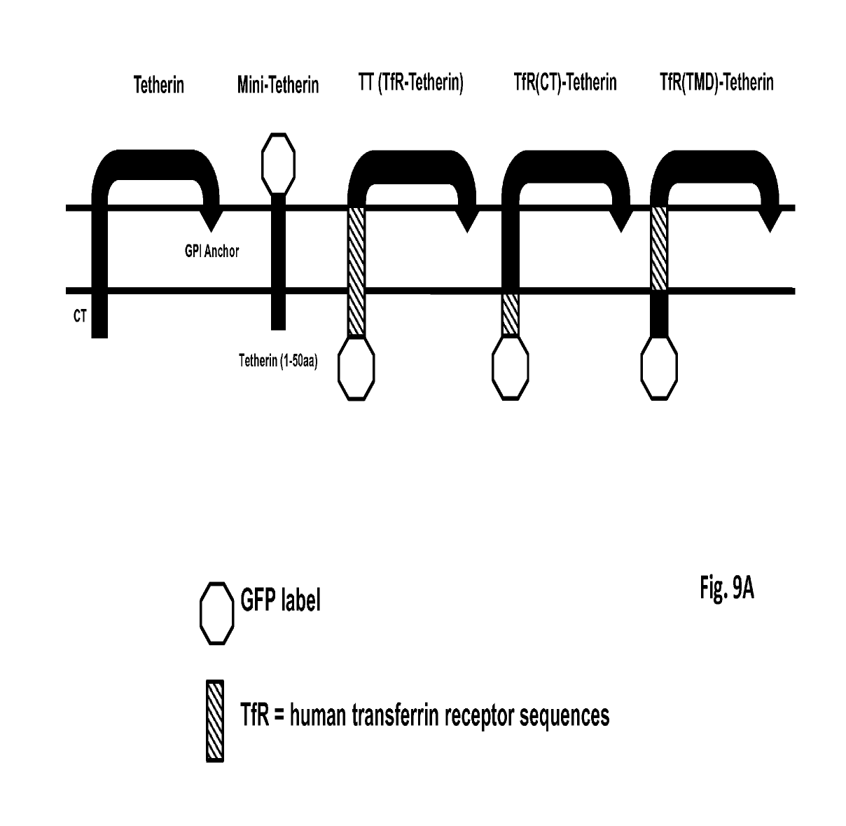

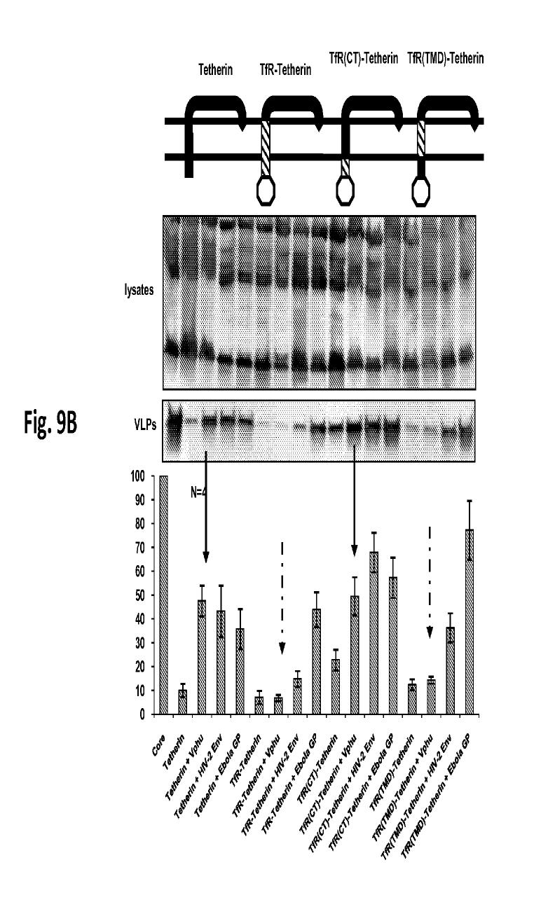

It is recognized herein that the particular Tetherin sequences for each species have specificity for particular anti-Tetherins from viruses that specifically infect those species. The comparison given in FIG. 1 guides those of skill in the art in selecting which residues to alter, if desired, for a given species, to reduce/abolish anti-Tetherin activity or to maintain anti-Tetherin activity. More specifically, a requirement for specific sequences in the Tetherin membrane spanning domain is shown by the fact that replacing this region with the equivalent region from the human Transferrin receptor protein (TfR) leads to a Tetherin derivative that retains the ability to block virus release, but is no longer counteracted by the HIV-1 Vpu anti-Tetherin protein (see the Examples below and FIG. 9). In addition, replacing the membrane spanning domain of human Tetherin with the equivalent region from the rhesus macaque Tetherin produces a Tetherin protein that retains the ability to block virus release, but is no longer counteracted by the HIV-1 Vpu anti-Tetherin protein, despite the substantial homology between these two sequences (data not shown). Similarly, replacing the cytoplasmic tail of Tethein with the region from TfR blocks the ability of KSHV K5 to counteract the protein (data not shown). Certain specific sequences in Tetherin are therefore required for the interaction with anti-Tetherin proteins, and the present disclosure provides those of skill in the art with the guidance needed to select mutations that achieve a desired goal.

A preferred reporter construct according to the invention comprises the transmembrane sequence of a Tetherin or a transmembrane sequence based on a Tetherin sequence. The transmembrane sequences of selected primate Tetherins are indicated in FIG. 1, although those of skill in the art will recognize that one or a few residues from either or both ends of the indicated region may be removed without significantly altering the activity discussed herein. A consensus transmembrane sequence is provided herein as SEQ ID NO:12.

An alternative preferred sequence for use in creating Tetherin reporter constructs according to the invention includes a transmembrane sequence coupled to a Tetherin cytoplamic tail. The cytoplasmic tail portion that can be included in embodiments of the invention includes residues that are typically present in soluble regions of a protein, and in particular, regions that are consistent with the cytoplasmic domain of type II membrane proteins. Those of skill in the art will thus immediately recognize that conservative mutations can be made within the cytoplasmic tail region of the Tetherin protein without abolishing activity. The comparison provided in FIG. 1 provides those of skill in the art with sufficient information to devise any number of cytoplasmic tail sequences for use in constructs according to the invention. A consensus cytoplasmic tail sequence is provided as part (residues 1-26) of SEQ ID NO:13, which shows a consensus sequence of the “stalk” region. Based on the information provided herein, those of skill in the art will immediately recognize the structures that should be maintained for embodiments that use a construct comprising a Tetherin cytoplasmic tail or a cytoplasmic tail derived from a Tetherin sequence. For example, mutation of one positively charged residue to another of the same charge can be made, mutation of one polar residue to another can be made, and mutation of one hydrophobic residue to another can be made. In addition, the cytoplasmic tail may also contain specific sequences that can be involved in interaction between a Tetherin and an anti-Tetherin, such as KSHV K5.

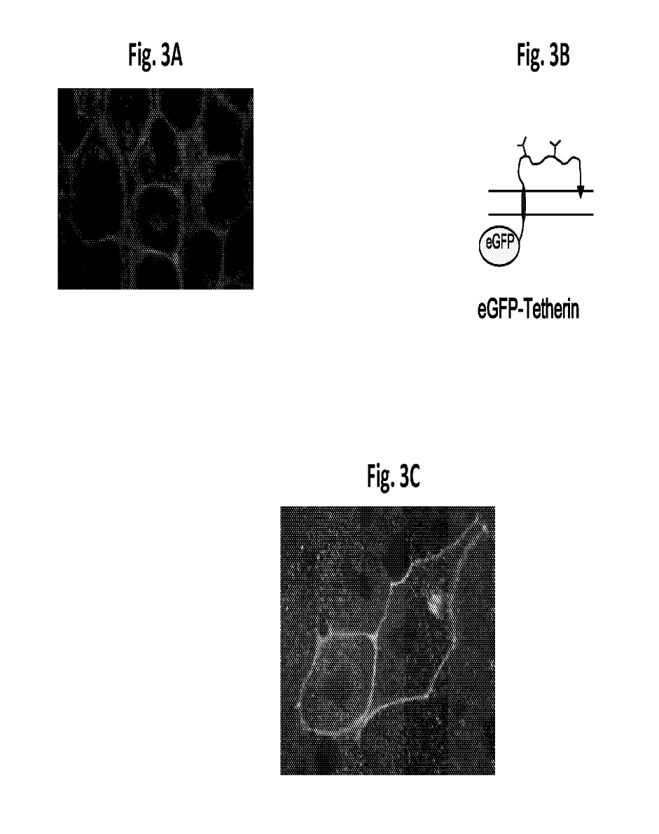

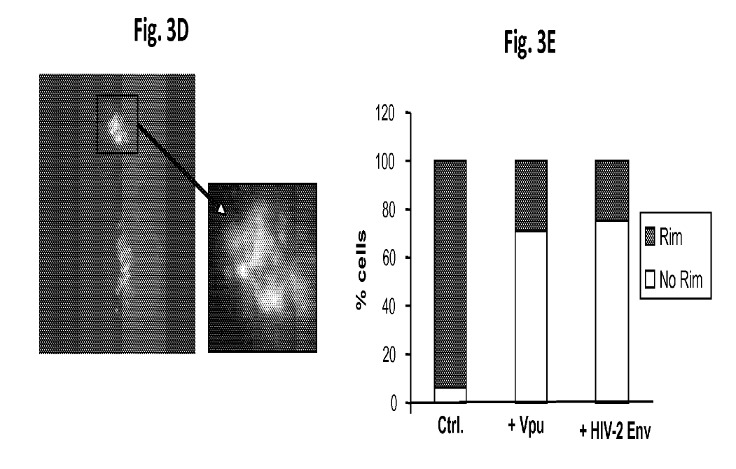

The transmembrane sequence and/or the cytoplasmic tail sequence can be based on any Tetherin desired. Due to the known homology between Tetherin sequences, those of skill in the art can easily devise alternative sequences to the wild-type sequences, while still retaining the desired activity. Initially, the cytoplasmic tail can be excluded from the constructs while still retaining activity. Furthermore, mutations at any of the regions of the cytoplasmic tail shown in FIG. 1 as not showing homology or conservation can be mutated (altered or deleted). Mutations that insert amino acid sequences in the cytoplasmic tail region can also be tolerated, as shown in the Examples below and, for example, FIG. 3, where a Green Fluorescent Protein is added to the N-terminus without affecting proper cellular localization and interaction with anti-Tetherins. It is to be recognized that the same concepts hold for sequences located C-terminal to the Tetherin transmembrane region.

One common way to express the amount of variation tolerated in making mutants is by way of percent identity comparisons. In embodiments, the present invention provides for Tetherin constructs that include a Tetherin transmembrane region showing about 43% or greater sequence identity, about 47% or greater sequence identity, about 59% or greater sequence identity, about 61% or greater sequence identity; about 66% or greater sequence identity, or about 94% or greater sequence identity to a wild-type Tetherin transmembrane sequence. Using the information in FIG. 1, those of skill in the art will know which residues can be varied without expecting to alter activity, and which types of mutations that should be made. As defining a transmembrane region with absolute precision is extremely difficult, it is to be understood that the above levels of identity can be based on any reasonable length of base sequence from about 17 residues to about 24 residues. Furthermore, those of skill in the art recognize that a high level of conservative changes to a sequence can be tolerated without abolishing activity. Thus, in general, any hydrophobic residue within the chosen transmembrane region can be altered to any other (or multiple residues, up to about three) hydrophobic residue. An additional structural feature that may be used to guide those of skill in the art in making mutations within a wild-type transmembrane region is the common secondary structural feature of an alpha-helix. The Tetherin transmembrane regions, like many others from other proteins, show a predominantly alpha-helical structure. Thus, those of skill in the art can use computer technology to determine if a contemplated alteration in the sequence will destabilize or destroy the alpha-helical structure of the region. Such alterations should be avoided.

In some exemplary embodiments, the Tetherin region of the Tetherin reporter construct comprises a Tetherin transmembrane domain (e.g., SEQ ID NO:12 and residues 23-43 of SEQ ID NO:10). In other exemplary embodiments, a Tetherin “stalk” of about 50 amino acid residues of Tetherin (e.g., SEQ ID NO:13 and residues 1-50 of SEQ ID NO:10), which includes both the transmembrane domain and cytoplasmic tail of Tetherin, comprises the Tetherin region of the reporter construct. As mentioned above, any number of variations of these sequences can be used.

The Tetherin reporter construct is a non-naturally occurring (i.e., artificial) product. It thus need not be provided in an isolated or purified form. However, in embodiments, it is provided as an isolated or purified (at least partially) form. It is also to be understood that non-human Tetherin proteins have homologous sequences to the human Tetherin. Thus, the concepts discussed above with regard to human Tetherin are equally applicable to Tetherins of other species. That is, the invention encompasses the use of the transmembrane domain of any Tetherin, preferably in conjunction with the cytoplasmic tail, as a feature of a reporter construct for detection of inhibition of Tetherin activity by an anti-Tetherin.

The Tetherin reporter construct also includes a region that provides for detection of the construct. While not so limited, in general, the “reporter region” comprises a polyamino acid, which can be, but is not necessarily, modified at one or more residues to provide advantageous properties. One non-limiting example of such modifications is the covalent linking of a detectable group (e.g., fluorescent tag, an immunogen) to an amino acid side chain. The reporter region can be or include any type of structure that is suitable for detection. It thus may be or include a protein or protein domain that is intrinsically detectable (e.g., fluorescence by any of the known fluorescent proteins, such as Green Fluorescent Protein, Red Fluorescent Protein, and the like), that can be detected through the use of a detection agent (e.g., by binding of an antibody, including a labeled antibody), or that has a biochemical activity that produces a detectable signal (e.g., an enzyme, such as luciferase). In some embodiments, the reporter region comprises the coding sequence for a protein that produces a detectable signal only when displayed outside a cell. For example, one or more immunogenic epitopes can be included in the reporter region, which can only be detected by antibodies when presented outside of the cell. Likewise, any of a number of detection systems that are commercially available, including those that use cell-impermeable detection reagents, can be used for the reporter region. While it is preferred that the reporter region of the Tetherin reporter construct be covalently linked to the Tetherin region, such as by peptide linkages, strong non-covalent associations between the two regions are also encompassed by the invention. The precise amino acid sequences of the various reporter regions is not critical to practice of the invention, and thus their sequences need not be disclosed herein. Data provided below shows that inclusion of such reporter regions does not alter production or proper cell-surface localization of the fusion constructs. Those of skill in the art thus may use any reporter region desired, with the expectation that the resulting reporter construct will function appropriately.

The reporter region of the reporter construct can be linked to the Tetherin region at the N-terminus of the Tetherin region or at the C-terminus of the Tetherin region. Because fusion at the C-terminus allows for localization of the reporter region outside of the host cell, it is preferred that the reporter region be located at the C-terminus of the Tetherin region. Of course, the Tetherin region and reporter region can be linked by way of one or more linker residues to provide various benefits. Those of skill in the art can easily select appropriate linker lengths and amino acid constituents for the linkers.

As will be apparent from the discussion above, applications of Tetherin reporter constructs, compositions comprising them, and methods using them may include in certain embodiments “mTX” constructs that can function as surrogates of the Tetherin/Anti-Tetherin interaction. The designation “mTX” can be described as follows: X is an enzyme, an antigen, or another reporter (signal) protein whose activity can be quantitated when expressed at the cell surface. Specific examples of X include, but are not limited to, β-lactamase and luciferase, whose enzymatic activity can be quantitated by adding specific substrates. Furthermore, as discussed in detail below, a stable cell line can express mTX plus an Ant-Tetherin, such as Vpu or K5 Inhibiting the mTX-Anti-Tetherin interaction leads to higher levels of mTX on the cell surface, and therefore greater X activity. This can be detected in an HTS format and the assay is therefore suitable for HTS of compounds to identify specific inhibitors of the Anti-Tetherin-Tetherin interaction.

The Tetherin reporter constructs of the invention can be purified or isolated substances, or can be part of compositions. Where the constructs are part of compositions, the compositions are not particularly limited. They thus can be any of a number of liquid or solid compositions, comprising any other substances or combination of substances. In general, it is preferred that the substances present in the composition in addition to the Tetherin reporter constructs are compatible with the stability and activity of the Tetherin reporter constructs. Non-limiting examples of additional substances include solvents, such as water, glycerol, or organic solvents (e.g., methanol), buffers (e.g., Tris, MOPS, HEPES), and salts (e.g., sodium salts, potassium salts, magnesium salts). Additional non-limiting substances that can be present in compositions according to the invention include some or all of the substances necessary for detecting the presence of the Tetherin reporter constructs. Non-limiting examples include antibodies, enzymatic substrates, energy (e.g., electron or electromagnetic radiation) donors for fluorescence, and energy acceptors/re-emitters. In some embodiments, the compositions comprise cells or cell lysates. Yet again, in some embodiments, the compositions comprise protein purification fractions.

While the Tetherin constructs of the invention have wide applicability for in vivo (including cell culture) production and detection, they can also be used in vitro in cell-free assays. Accordingly, in some embodiments, compositions comprising the Tetherin reporter constructs include purified or isolated Tetherin reporter constructs in combination with in vitro detection reagents. In some embodiments suitable for use in vitro the Tetherin reporter construct or one or more detection reaction components is immobilized, for example on insoluble beads or on a membrane.

The Tetherin reporter constructs of the invention are generally encoded by nucleic acids. The nucleic acids of the invention comprise engineered sequences that encode a Tetherin-derived sequence that is sufficient for both cell-surface membrane localization and anti-Tetherin interaction. This nucleic acid sequence is fused in-frame to a coding sequence for a polyamino acid that can participate in a detection system. Standard, widely practiced methods of making fusion nucleic acids can be used to create the nucleic acids of the invention. In accordance with the discussion above, the polyamino acid sequence can have an intrinsic detectable activity or signal, can be post-translationally modified to provide a detectable activity or signal, or can participate in a reaction that produces a detectable activity or signal.

In embodiments, the nucleic acid of the invention consists of the coding sequence of a Tetherin reporter construct. In embodiments, the nucleic acid of the invention comprises the coding sequence of a Tetherin reporter construct, wherein the sequence includes the coding region and additionally includes one or more nucleotides at either or both ends of the coding sequence. In preferred embodiments, the nucleic acid comprises some or all of the regulatory elements required for expression of the Tetherin reporter construct in a chosen host cell. It thus may comprise promoters, transcription factor binding sites, and the like. Any number and combination of expression control elements may be included in the nucleic acids, and those of skill in the art are free to select appropriate and/or desired elements based on the particular intended use of the Tetherin reporter construct.

In embodiments, the nucleic acid is a vector for introduction and/or maintenance of the nucleic acid in a host cell. For example, the nucleic acid may be a plasmid suitable for insertion into a host cell and production of a Tetherin reporter construct. Numerous vector backbones are known and commercially available, and any suitable vector backbone may be used in accordance with the present invention. Preferably, the vector is capable of being maintained in a host cell at least long enough to express the Tetherin reporter construct. In some embodiments, at least the coding region, more preferably the coding region plus expression control sequence(s), are stably inserted into the genome of a host cell. Thus, in embodiments, the nucleic acid is an engineered genome of a host cell. Where intended for insertion into a genome of a host cell, the nucleic acid can comprise one or more sequences for insertion into the host cell genome. For example, the nucleic acid can comprise insertion element sequences, viral insertion sequences, or sequences designed for homologous recombination at a specific site in a host genome.

As with other embodiments of the invention, because the nucleic acids of the invention encode non-naturally occurring proteins, the nucleic acids are likewise non-naturally occurring. In certain embodiments, the nucleic acids are purified or isolated from other substances, such as cellular molecules. Furthermore, the nucleic acids of the invention are not limited to any particular type of nucleic acid, but rather can be any type. The nucleic acids thus may be double-stranded DNA, single-stranded DNA, double-stranded RNA, single-stranded RNA, or a modified form of DNA, such as PNA. The nucleic acids thus may be mRNA or a nucleic acid derived therefrom, such as cDNA.

The nucleic acids of the invention include coding sequences for the Tetherin reporter constructs of the invention. Exemplary amino acid sequences for the Tetherin region of the reporter constructs are provided above and can be found in the literature. For example, the nucleic acid sequence can be taken from GenBank Accession Numbers: NM_{—}004335, FJ943431, FJ345303, FJ868941, CJ479048, DY743778, and XP_{—}512491. Alternatively, the nucleic acid sequence can be a nucleic acid sequence according to SEQ ID NO:8, which provides a nucleic acid sequence encoding the consensus sequence of SEQ ID NO:12, or a nucleic acid according to SEQ ID NO:9, which provides a nucleic acid sequence encoding the consensus sequence of SEQ ID NO:13. Of course, due to the degeneracy of the genetic code, alterations in the precise sequences of SEQ ID NO:8 and SEQ ID NO:9 can be made without altering the encoded amino acid sequences. In embodiments, the nucleic acids of the invention show 50% or more sequence identity with SEQ ID NO:8 or SEQ ID NO:9, as calculated over the length of SEQ ID NO:8 or SEQ ID NO:9 only (i.e., not calculated based on the entire sequence of the full construct). In embodiments, the level of sequence identity is about 75% or more, about 90% or more, about 95% or more, about 97% or more, or about 99% or more. Those of skill in the art are to understand that each particular value falling within 50% to 100% (e.g., 51%, 52%, 53%, etc.) is specifically envisioned as a value according to the invention, and the need to recite each particular value is not necessary to capture this subject matter. Those of skill in the art can derive suitable nucleic acid sequences to encode such amino acid sequences, based on the genetic code, with ease. For example, publicly available computer programs can be used to reverse translate the polyamino acids provided herein to arrive at exemplary nucleic acids according to the invention. Likewise, those of skill in the art can make suitable nucleic acids using standard molecular biology techniques. Because those of skill in the art are fully capable of producing all of the nucleic acids encompassed by the present invention, each particular sequence need not be disclosed herein.

The nucleic acids of the invention can comprise, in addition to the coding sequence for a Tetherin reporter construct, the coding sequence for an anti-Tetherin. Providing both coding sequences on the same nucleic acid ensures that the same number of copies of each coding sequence is introduced into a host cell. In this way, variation in expression of the two proteins can be minimized. The two coding sequences can be expressed from the same promoter/operator region. Alternatively, each coding sequence can be expressed independently, from different promoter/operator regions. While any suitable elements for expression control may be used, and the expression control elements selected independently for each coding sequence, it is preferred that the same control elements be selected to better ensure that an optimal expression of the encoded proteins occurs. The sequences of various anti-Tetherins are publicly available; thus, those of skill in the art can make and use such sequences with ease.

The invention also provides recombinant cells (also referred to herein as host cells) comprising the Tetherin reporter constructs, nucleic acids encoding them, or both. The recombinant cells of the invention can be any cell of interest to the practitioner. The cell thus may be a bacterial cell for production or amplification of a plasmid or other vector comprising a nucleic acid of the invention. Alternatively, the cell can be a prokaryotic or eukaryotic cell for production of Tetherin reporter constructs and/or anti-Tetherin proteins. Yet again, the recombinant cell can be a eukaryotic cell for use in an assay of the invention (discussed in detail below). Recombinant cells can be used for protein or nucleic acid production purposes, for research purposes, or for drug discovery purposes.

In exemplary embodiments, the recombinant cells of the invention are used in assays for detection of inhibition of Tetherin cell-membrane localization. The recombinant cells of the invention may have normal, reduced, or no wild-type, full-length, endogenous Tetherin expression, but optimal cell types minimize interference with assays performed using the cells. Numerous cells that do not express Tetherin are known in the art, and any such cells can be used. In exemplary embodiments, human HeLa cells are used. Alternatively, a cell that naturally expresses Tetherin can be engineered to show reduced or no Tetherin expression. The gene for human Tetherin is known, as are assays for its production and activity. Those of skill in the art will immediately recognize that, using random mutagenesis or specific mutagenesis using the human Tetherin sequence as a guide, mutations in the genomic Tetherin gene can be made, which will reduce or abolish its production and/or activity. Such techniques are routine in the art.

While any cell may be used as a host cell, it is preferred that the cell be of the same species as the Tetherin region of the Tetherin reporter construct and/or be a cell of a species in which the anti-Tetherin of interest can be found in nature. Thus, for example, where the practitioner is interested in the interaction of human Tetherin with an HIV anti-Tetherin, it is preferred that human cells be used as host cells. Even more preferably, the particular cell type (e.g., T cells, macrophages) in which a natural interaction between a Tetherin and anti-Tetherin occurs is used as the host cell.

An additional general aspect of the invention relates to assays for identification of interaction between a Tetherin reporter construct and an anti-Tetherin. The Tetherin reporter constructs of the invention possess sequences (structures) for both cell-surface localization and for inhibition of cell-surface localization by an anti-Tetherin. They also possess the ability to produce, or participate in the production of, detectable signals, which indicate the cellular location of the constructs or presence of functional Tetherin constructs. It is known that anti-Tetherins have the ability to block proper cell-surface localization of Tetherins, remove from a cell surface Tetherins already present there, or degrade Tetherins so they cannot function. As such, the cellular localization of the Tetherin reporter constructs provide a simple and powerful way to determine whether an anti-Tetherin has inhibited the proper cellular location of a Tetherin construct. Specifically, according to the method of the invention, substantial localization of a Tetherin reporter construct on the cell surface of a cell indicates lack of inhibition by an anti-Tetherin, whereas a lack of substantial localization on the cell surface indicates inhibition by the anti-Tetherin. In some embodiments, the anti-Tetherin retains the Tetherin construct in the cytoplasm, where it can be detected in some situations. In other embodiments, the anti-Tetherin degrades the Tetherin construct, and no detectable signal is produced. Thus, in some embodiments, the method includes not only determining if a signal is present on a host cell's surface, but further includes determining if a signal is present in the cytoplasm of a host cell.

In general, the method of this aspect of the invention comprises exposing a Tetherin reporter construct to an anti-Tetherin and determining whether the anti-Tetherin inhibits the proper localization of the Tetherin reporter construct on the cell surface. In exemplary embodiments, the method comprises: expressing a Tetherin reporter construct in a cell that expresses an anti-Tetherin; and determining the cellular location of the Tetherin reporter construct. Substantial presence of the Tetherin construct at the cell surface indicates an inability of the anti-Tetherin to inhibit cell-surface localization of the Tetherin construct, whereas substantial cytoplasmic presence of the Tetherin construct indicates the ability of the anti-Tetherin to inhibit cell-surface localization. Expression of the anti-Tetherin can be from any source, including viral nucleic acids (e.g., modified HIV viruses), exogenously supplied vectors (e.g., plasmids), or from the same nucleic acid that the Tetherin reporter construct is expressed.

The method of the invention can take many forms, but in a preferred embodiment, the method is a method of identifying one or more substances that reduce or abolish the ability of an anti-Tetherin to inhibit cell-surface localization of a Tetherin reporter construct. According to the method, expression of both a Tetherin construct and an anti-Tetherin results in substantial, if not complete, cytoplasmic localization of the Tetherin reporter construct. Introduction (by any means) of a substance that inhibits the activity of the anti-Tetherin on the Tetherin reporter results in substantial, if not complete, localization of the Tetherin reporter construct on the cell surface. The cellular localization can thus be used as an indicator of the substance's ability to inhibit the anti-Tetherin activity. The method thus may comprise: exposing a Tetherin reporter construct to an anti-Tetherin in a cell; allowing adequate time for the two to interact; exposing the Tetherin reporter construct and anti-Tetherin to one or more substances (which can be independently added or added as part of a sample or composition); and determining the cellular location of the Tetherin reporter construct.

The substance that inhibits anti-Tetherin activity can do so by way of any molecular mechanism. It thus may be a substance that binds the Tetherin reporter construct and blocks access to the construct by the anti-Tetherin. Alternatively, it may be a substance that binds the anti-Tetherin and blocks its ability to bind the Tetherin construct. Yet again, it may be a substance that binds an additional member of a Tetherin construct/anti-Tetherin complex (or an intermediary in the inhibitory process that does not involve a complex). The precise mechanism of action of the substance is not critical to performance of the method, as the method will be capable of detecting the cellular location of the Tetherin reporter construct regardless of the particular molecular mechanism of action of anti-Tetherin on the Tetherin reporter construct.

The method of the invention can be a method of drug discover or of discovery of lead compounds for generation of drugs. In embodiments, the method is a method of identifying anti-viral agents, such as those that can inhibit the infection, propagation, and/or spread of enveloped viruses that utilize an anti-Tetherin to combat the host's response to infection. The anti-viral agents can be targeted against many different viruses. One non-limiting example is anti-HIV agents.

The methods of the invention can include positive and/or negative controls to improve confidence levels. For example, for each sample/compound(s) tested, one may also perform a similar assay, but lacking one or more components. One example of a positive control for expression of the Tetherin reporter construct includes expressing the Tetherin construct in the absence of the anti-Tetherin. A positive result would indicate that the Tetherin reporter construct is being expressed properly and at an adequate level for detection. Yet another example of a control is to express the Tetherin reporter construct in the absence of an anti-Tetherin, expose the cell to a sample/substance(s), and determine the cellular location of the Tetherin construct. Localization at the cell surface indicates that the sample/substance(s) do not directly inhibit proper cellular localization of the Tetherin construct. Those of skill in the art are fully capable of developing additional or alternative control reactions to assist in assay read-out without undue experimentation and without the need for each control to be specified herein.

In one embodiment of the method, the step of determining the cellular location of the Tetherin reporter construct can be repeated. For example, a method may include expressing a Tetherin construct followed by determining its cellular location, then expressing an anti-Tetherin and determining if a change in the cellular location of the Tetherin construct occurred.

The method of the invention can be a high-throughput screening (HTS) method for identifying bioactive agents (e.g., drugs) having anti-Tetherin inhibitory activity. The methods thus may be methods of identifying anti-viral agents, which can be used in therapeutic treatments. HTS assay parameters are known in the art, and the general principles of HTS assays can be applied to the present invention. In general, the HTS assay of the invention comprises maintaining cells in multiple assay containers (e.g., wells of a microtiter plate), expressing a Tetherin reporter construct and an anti-Tetherin in the cells, exposing the cells to one or more substances, and determining the cellular localization of the Tetherin reporter construct. According to the HTS assay, a large number of substances can be screened at a single time for anti-Tetherin inhibitory activity. The HTS method can include screening a panel of compounds (e.g., 10, 100, 1000) per well. Wells showing positive results (i.e., cell surface localization of the Tetherin construct) can then be re-screened using fewer compounds per well until a single or a few bioactive substances are identified.

The method of the invention, by using a detectable signal as a readout, is highly amenable to HTS assays, particularly when the readout is a visual or optical result. Numerous instruments are commercially available for detection of optical signals, and any of those can be used in conjunction with the assay. For example, automated confocal microscopy or fluorescent plate readers can be used to detect a fluorescent signal and determine the cellular location of the origin of the signal, i.e., whether on the cell surface. Some readouts might only detect expression if the construct is on a cell surface, e.g., enzyme assays or detection systems that use a cell-impermeable substrate. Alternatively, flow cytometry (e.g., FACS) can be used.

One advantage that the present HTS methods provide is the ability to screen for cells that exhibit the largest dynamic range of activity following inhibition of the anti-Tetherin-Tetherin interaction from, for example, the addition of anti-Vpu siRNAs. For example, testing compounds against matched Vpu expressing and non-expressing Tetherin-reporter cell lines is used to reveal any non-specific effects of test compounds. The quality of the assay can be evaluated by statistical analysis using both the variation coefficients for the positive and negative control replicates and Z′ scores. The Z′ score is a parameter for the success of a HTS assay, where Z′=1-(3σ_{c+}+3σ_{c-})/(3μ_{c+}−3μ_{c-}). This measure depends on the sums of the standard deviations of the positive and negative controls, as well as the difference between the mean activities of those controls. Z′ scores within acceptable limits (greater than 0.5, and optimally close to 1.0) will validate the assay for HTS. In this effort, one ensures that assay results are stable over the entire plate, between plates, between days, and between reagent batches (e.g., cell line batches).

HTS can be used to screen chemical libraries with compounds in pilot screens assayed at a single concentration, typically 10 μM final compound concentration. Hits are defined using standard criteria (either 3 or 6 standard deviations from the mean value of the negative control samples depending on assay performance and hit rate). Based on these criteria, one can identify compounds with EC₅₀ values ≦20 μM, and typically in the 1-10 μM range. After the first pass screen, hits are picked into a single plate and a reconfirmation screen is performed. This can be followed by more specific evaluations of inhibitor activity using endogenous Tetherin activity, in VLP release assays from HeLa cells, and by evaluating the effect on HIV-1 replication in human T cells lines.

It is to be noted that each particular Tetherin can have one or more anti-Tetherins that are specific for interaction. Those of skill in the art are aware of the various combinations of Tetherins and anti-Tetherins that can be used in combination, or can easily identify appropriate combinations. An exhaustive list of Tetherin and anti-Tetherin pairings thus need not be provided here.

In yet another general aspect, the invention provides kits. The kits of the invention can take many forms and configurations, but in general include one or more of the constructs, nucleic acids, or cells of the invention. Kits can include some or all of the biological or chemical components required to practice a method of the invention. At its basic level, a kit of the invention includes one or more containers containing the constructs, nucleic acids, and/or cells of the invention. Where two or more containers are provided, they are provided in packaged combination, for example in a box. Each container may comprise one of the products of the present invention, or may contain two or more of the products. For example, a container may contain a plasmid encoding a Tetherin reporter construct, or it may contain two different plasmids, one encoding a Tetherin reporter and the other encoding an anti-Tetherin. Likewise, a kit may include one or more containers containing a plasmid encoding a Tetherin reporter construct and an anti-Tetherin, and additionally include one or more containers containing cells suitable for transformation or transfection with the plasmid. The cells may, of course, be competent cells ready for transformation or transfection. In a preferred embodiment, the kit comprises multiple containers, each containing recombinant cells that have within them the coding regions for a Tetherin reporter construct and an anti-Tetherin. Such recombinant cells are immediately useful for performing an assay according to the invention, such as an assay for identification of drugs.

The kits of the invention can include some or all of the reagents and equipment for setting up and performing an assay according to the invention. The kits thus may include one or more microtiter plates; media for growth of recombinant cells; antibodies, enzymatic substrates, or other reagents for detection of the Tetherin reporter constructs; and/or cells (recombinant or natural) for performing control reactions; and reagents for positive and negative controls.

### EXAMPLES

The invention will be further explained by the following Examples, which are intended to be purely exemplary of the invention, and should not be considered as limiting the invention in any way.

### Example 1

### Production and Use of a Tetherin Reporter Construct

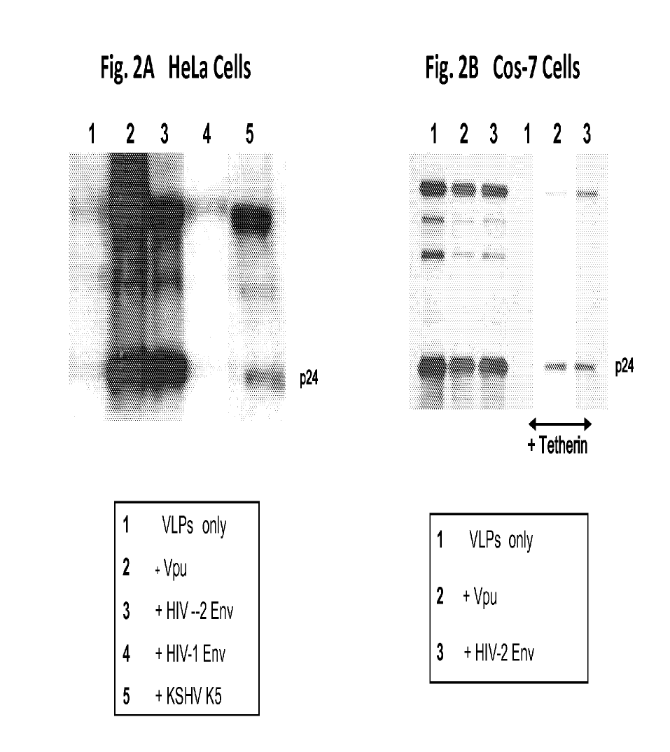

Human Tetherin restricts HIV-1 release, and this restriction can be overcome by HIV-1 Vpu, HIV-2 Env, and KSHV K5. Human (HeLa) cells restrict the release of HIV-1 virus-like particles (VLPs), while simian (Cos-7) cells do not (Noble et al., 2006, J. Virol. 80:2924-2932; Varthakavi et al., 2003, Proc. Natl. Acad. Sci. USA 100:15154-15159). The basis for this restriction is the human Tetherin/Bst-2 protein (Neil et al., 2008, Nature 451:425-430; Van Damme et al., 2008, Cell Host. Microbe 3:245-252). It is shown herein that HIV-1 Vpu and the HIV-2 Env can counteract this restriction and thereby increase the level of VLPs released from HeLa cells, and it has now been determined that KSHV K5 shares this activity (FIG. 2A). More specifically, FIG. 2A shows Western blot results of transfection of HeLa cells with an HIV-1 Gag-Pol-Rev plasmid, which results in the release of VLPs that can be pelleted from supernatants and detected by Western blotting with anti-p24 antisera. FIG. 2A shows Western blot results indicating that VLP release from Tetherin-positive HeLa cells is increased by co-expression of HIV-1 Vpu, HIV-2 Env, or KSHV K5, but not by the HIV-1 Env. Furthermore, FIG. 2B shows that adding human Tetherin to Cos-7 cells severely inhibits VLP release, and this can be partially overcome by both HIV-1 Vpu and HIV-2 Env.

As discussed above, endogenous Tetherin in HeLa cells is mostly present at the cell surface, with some intracellular puncta (see FIG. 3A), and HIV-1 Vpu and HIV-2 Env remove Tetherin from the surface of cells. To facilitate visualization of Tetherin, a derivative of human Tetherin with an N-terminal fusion of an enhanced Green Fluorescent Protein (eGFP) reporter (which is therefore expressed in the cytoplasmic domain of this Type II membrane protein) was constructed. The construct is generally depicted in cartoon form in FIG. 3B. When transfected into HeLa, Cos-7 or Human Embryonic Kidney cells (293 cells), eGFP-Tetherin was observed at both the cell surface and an intracellular location that overlapped with Golgi/TGN markers (FIG. 3C). Co-expression of HIV-1 Vpu or HIV-2 Env dramatically altered the distribution of eGFP-Tetherin, removing the cell surface rim and increasing the intensity of the Golgi/TGN concentration (FIG. 3D). The data is presented in bar graph form in FIG. 3E. These data suggested that Tetherin restricts HIV release through an interaction with virions at the cell surface, and that HIV's anti-Tetherin factors, Vpu and Env, act, at least in part, by removing Tetherin from the plasma membrane. The data also show that a labelled Tetherin protein can be used to identify the cellular location of the Tetherin, and to monitor the effects of anti-Tetherins on the Tetherin.

### Example 2

### Production and Use of an Alternative Tetherin Reporter Construct

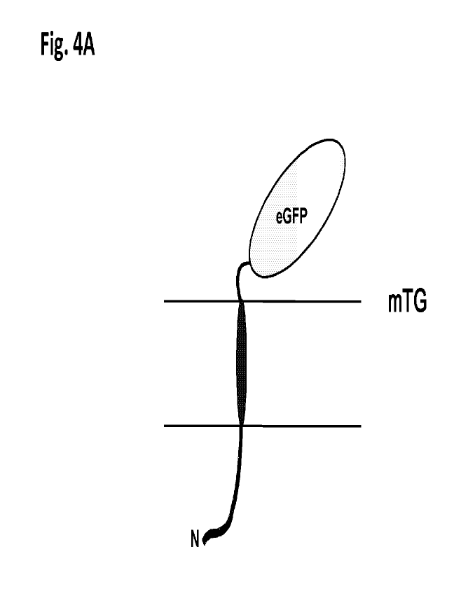

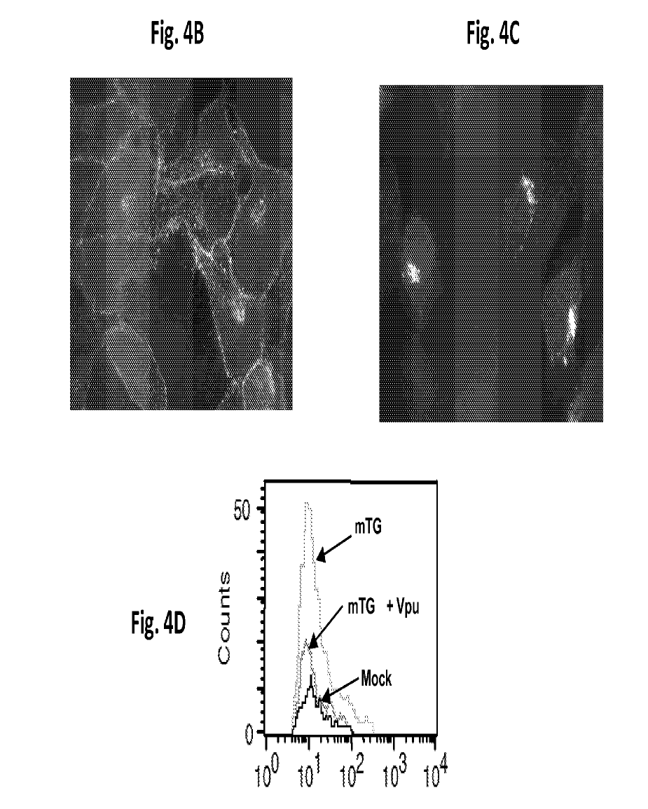

The human Tetherin protein was partially dissected to determine regions that are important for cellular localization and inhibition by anti-Tetherins. A fragment of human Tetherin was generated using the published sequence of Tetherin. The fragment included only the N-terminal 50 amino acid residues of the full-length protein. As can be seen from FIG. 4A, the construct included an N-terminal Tetherin-derived cytoplasmic tail, a Tetherin-derived transmembrane domain, and an extracellular GFP protein. It also included a two residue glycine (G-G) linker between the transmembrane domain of Tetherin and the eGFP protein. This construct was called “mTG” for “mini-Tetherin-GFP”. Its general structure is depicted in cartoon fashion in FIG. 4A and its sequence is provided as SEQ ID NO:10.

Experiments showed that the 50 amino acid residue “stalk” of Tetherin is sufficient to direct a reporter protein (eGFP) to the cell surface. It was also surprisingly found that the same 50 residue fragment was sufficient to confer sensitivity to the anti-Tetherins Vpu and K5. More specifically, two experiments were performed. In the first experiment, shown in FIG. 4B, 300 ng of plasmid encoding the mTG construct (pCMV6-XL5-BST-2-GFP) was transfected into an 80-90% confluent 10-cm plate of HeLa cells using Lipofectamine 2000 (Invitrogen, Carlsbad, Calif.). At 24 hours post-transfection, cells were transferred to coverslips coated with poly-L-lysine (Sigma-Aldrich, St. Louis, Mo.). After an additional 24 hours, cells were fixed with 4% paraformaldehyde for 20 minutes at room temperature, washed three times in phosphate buffer saline (PBS) and permeabilized for 20 minutes in 0.1% Triton X-100 at room temperature, followed by three additional PBS washes. Primary antibody staining was performed using a mouse anti-GFP monoclonal antibody (Invitrogen) at a 1:500 dilution for 1 hour at room temperature. A donkey anti-mouse AlexaFluor 488 secondary antibody (Invitrogen) was used at 1:200 dilution for 1 hour at room temperature. Processed cells were mounted in Prolong Gold antifade reagent with DAPI (Invitrogen). The cellular localization of the mTG construct was analyzed using the Perkin Elmer Ultraview ERS laser spinning disk confocal imaging system at 100× magnification (PerkinElmer, Waltham, Mass.). Images were acquired using this microscope and processed using Volocity software (Improvision, PerkinElmer) and Adobe Photoshop Creative Suite 2.

In the second experiment, shown in FIG. 4C, 300 ng of a plasmid encoding the mTG construct was co-transfected into HeLa cells together with 2 ug of a plasmid encoding HIV-1 Vpu (pVphu-HcRed). Transfected cells were processed for confocal analysis as described above. The Vpu protein used contains a C-terminal HcRed tag, which allowed visualization of Vpu without the need for specific anti-Vpu antibodies. As can be seen from the figure, the presence of Vpu removed mTG from the cell surface and concentrated it at an intracellular location.

As can be seen in the confocal micrograph of FIG. 4B, the mTG construct was expressed and localized to the cell surface, as well as intracellular puncta to some extent. Because its cellular localization is essentially identical to full-length Tetherin, the 50 residue stalk is a suitable surrogate for full-length Tetherin in assays to detect cellular localization. Interestingly, co-transfection and expression of HIV-1 Vpu substantially blocked localization of mTG at the cell surface (see the confocal micrograph of FIG. 4C). Similar results were obtained for co-transfection of mTG and K5. The construct is thus also a suitable surrogate for full-length Tetherin in assays to detect anti-Tetherin activity.

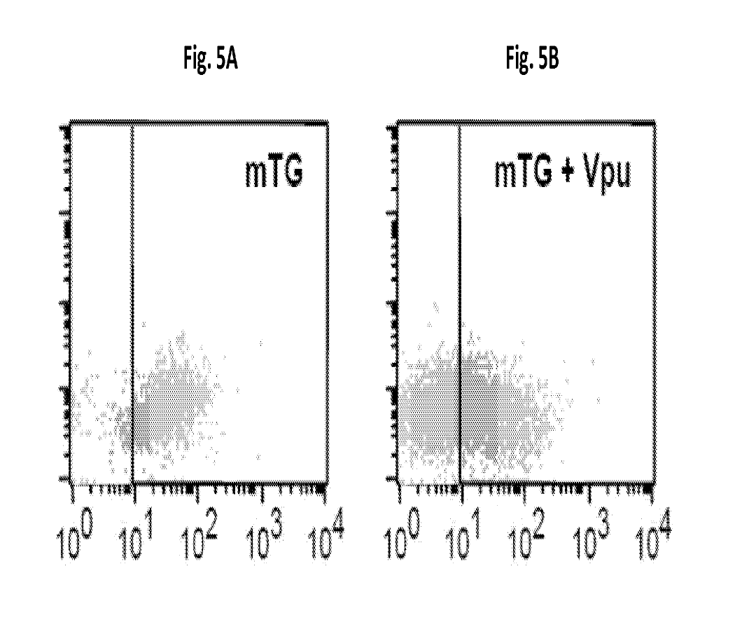

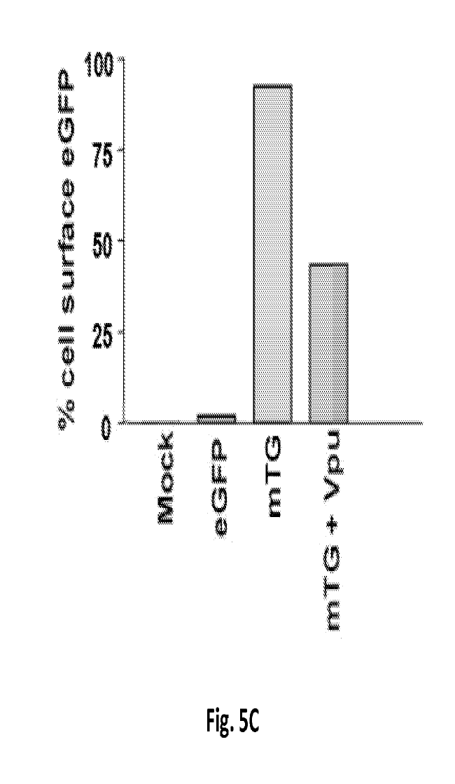

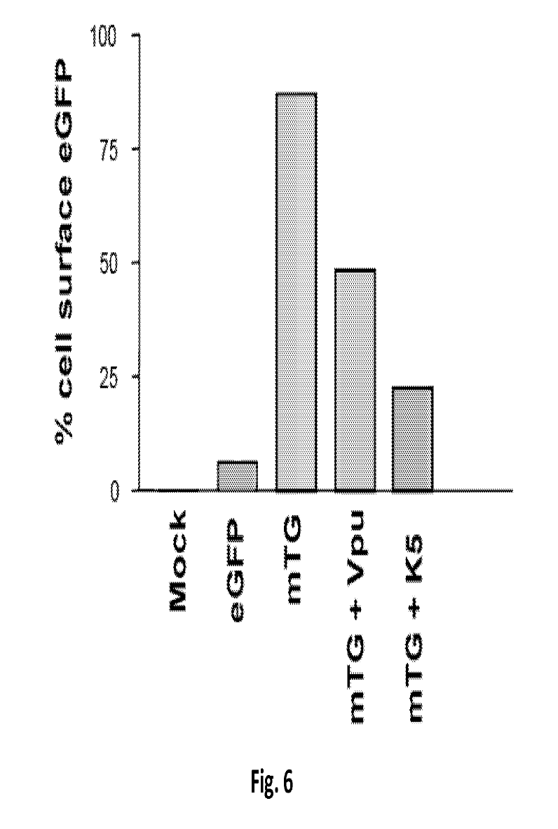

The results provided in FIGS. 4B and 4C were confirmed using FACS analysis, and the results of the confirmatory experiments are presented in FIG. 5, Panels A-C. FACS analysis to confirm cell surface expression of mTG was determined in HeLa cells transfected with 1 ug of mTG, or co-transfected with 1 ug of mTG and 4 ug of either Vpu (plasmid pVphu) or KSHV K5 (plasmid pTracer-K5). 24 hours post-transfection, cells were resuspended in blocking buffer (0.5% bovine serum albumin in PBS) and incubated 20 minutes at 4° C. To detect mTG directly, a conjugated anti-GFP-AlexaFluor 647 antibody (Invitrogen) was used at a 1:200 dilution. Cells were washed twice with 1 ml of PBS, resuspended in 400 ul of 4% paraformaldehyde, and analyzed on a FACSCALIBUR flow cytometer (BD, Franklin Lakes, N.J.). Cells expressing mTG are shown in the region of the plot to the right of the perpendicular line (FIGS. 5A and 5B). This data can also be shown graphically by plotting the % of cells expressing cell surface eGFP by calculating the % of cells in this compartment. Such representations are shown in FIGS. 5C and 6. The data show that mTG can be down-regulated from the cell surface by the co-expression of Vpu (FIG. 5) or K5 (FIG. 6).

### Example 3

### Construction of Tetherin Reporter Construct Containing Luciferase

To further show the versatility and broad applicability of the constructs of the present invention, a Tetherin reporter construct was constructed, which contained the N-terminal 50 residues of human Tetherin fused to Gaussia luciferase. The Gaussia luciferase gene was amplified by PCR from the pCMV-GLuc plasmid (New England Biolabs) and fused to the mini-Tetherin sequences in the same was as mT was constructed. The construct is referred to as mTL (pCMV6-XL5-BST-2-GLuc). The cellular localization of mTL was determined by confocal microscopy. Confluent HeLa cells were transfected with either 300 ng of plasmid encoding the mTL construct alone, or together with 2 ug of HIV-1 Vpu (pVphu-HcRed) using Lipofectamine 2000 (Invitrogen, Carlsbad, Calif.). At 24 hours post-transfection, cells were transferred to coverslips coated with poly-L-lysine (Sigma-Aldrich, St. Louis, Mo.). After an additional 24 hours, cells were fixed with 4% paraformaldehyde for 20 minutes at room temperature, washed three times in phosphate buffer saline (PBS), and permeabilized for 20 minutes in 0.1% Triton X-100 at room temperature, followed by three additional PBS washes. Primary antibody staining was performed using a polyclonal rabbit anti-Gaussia luciferase antibody (Nanolight Technology, Pinetop, Ariz.) at a 1:1000 dilution for 1 hour at room temperature. A donkey anti-rabbit AlexaFluor 488 secondary antibody (Invitrogen) was used at 1:200 dilution for 1 hour at room temperature. Processed cells were mounted in Prolong Gold antifade reagent with DAPI (Invitrogen). The cellular localization of the mTL construct with or without the co-expression of Vpu was analyzed using the Perkin Elmer Ultraview ERS laser spinning disk confocal imaging system at 100× magnification (PerkinElmer, Waltham, Mass.). Images were acquired using this microscope and processed using Volocity software (Improvision, PerkinElmer) and Adobe Photoshop Creative Suite 2.

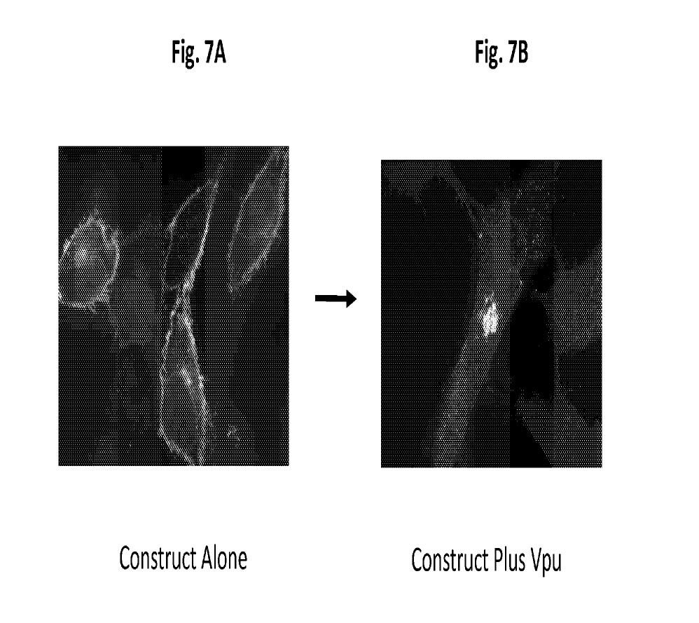

As can be seen from FIG. 7, Panel A, expression of the construct led to localization of the mTL construct at the cell surface. Panel B shows that co-expression with HIV-1 Vpu eliminates the cell-surface localization. The mTL construct is thus fully capable of recapitulating the activity of Tetherin with respect to an anti-Tetherin, and thus is a suitable construct for use in assaying for Tetherin inhibition by anti-Tetherins and for identification of modulators of that inhibition.

Similar results can be obtained using the Vargula luciferase. In addition, similar results were obtained using β-lactamase, whose activity can be detected by the cleavage of colorimetric or fluorogenic substrates containing β-lactam rings.

These constructs, like others of the invention, allow their activity to be measured without requiring cell lysis, or transfer of supernatants to fresh wells. In addition, the use of reporter enzymes (e.g., luciferase, alkaline phosphatase) and their specific substrates obviates the need for multiple incubations and/or washing steps as, for example, can be needed with cell-free antibody based systems. It is to be recognized, though, that embodiments of the invention include use of antibodies (e.g., anti-GFP, anti-luciferase) to detect cell surface expression. In these embodiments, the ease of use and availability of commercial antibodies provides an advantage. Further advantages provided by embodiments of the invention include the use of microscopy to detect distribution in cells (e.g., for GFP constructs) and overall signal detection by plate readers (e.g., for GFP or HaloTag™; data not shown).

### Example 4

### Construction of Tetherin Reporter Construct Containing HaloTag™

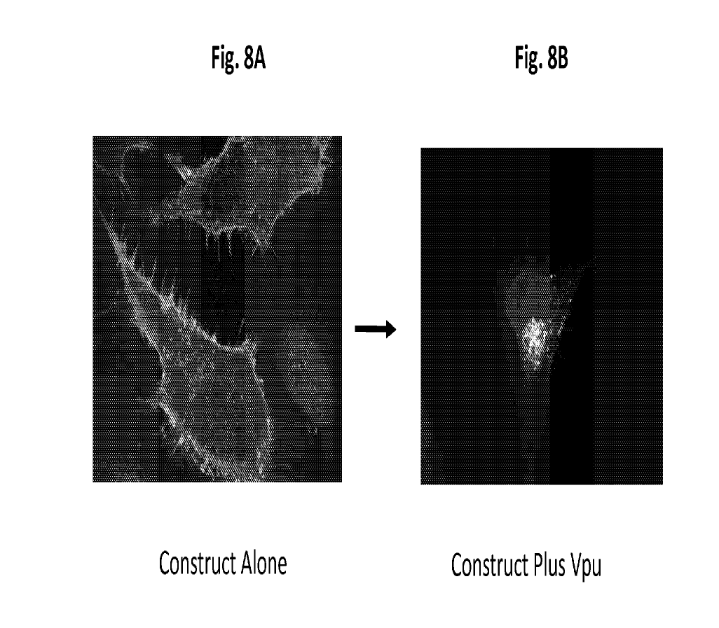

To yet further show the versatility and broad applicability of the constructs of the present invention, a Tetherin reporter construct was created that contained the N-terminal 50 residues of human Tetherin fused to a HaloTag™ (Promega, Madison, Wis.) reporter region. The construct is referred to herein as mTH. More specifically, the mTH construct was made by using PCR to amplify and fuse the first 50 residues of Tetherin to the HaloTag™ gene from plasmid pFC14A(HaloTag™)-CMV Flexi™ Vector (Promega, Madison, Wis.). The HaloTag™ protein is a 33 kD monomeric protein which is not endogenous to mammalian cells and can be detected with an anti-HaloTag™ antibody or the HaloTag™ ligand. The HaloTag™ ligand is membrane impermeable and is tagged with a fluorophore, AlexaFluor 488. The ligand covalently binds the HaloTag™, in this case mTH expressed on the cell surface. In FIG. 8, HeLa cells were transfected with 300 ng of mTH alone (FIG. 8A), or co-transfected with Vpu (pVphu-HcRed) (FIG. 8B) using Lipofectamine 2000 (Invitrogen). At 24 hours post-transfection, cells were transferred to coverslips coated with poly-L-lysine (Sigma-Aldrich, St. Louis, Mo.). After an additional 24 hours, cells were fixed with 4% paraformaldehyde for 20 minutes at room temperature, washed three times in phosphate buffer saline (PBS) and permeabilized for 20 minutes in 0.1% Triton X-100 at room temperature, followed by three additional PBS washes. Primary antibody staining was performed using a polyclonal rabbit anti-HaloTag™ antibody (Promega, Madison, Wis.) at a 1:500 dilution for 1 hour at room temperature. A donkey anti-rabbit AlexaFluor 488 secondary antibody (Invitrogen) was used at 1:200 dilution for 1 hour at room temperature. Processed cells were mounted in Prolong Gold antifade reagent with DAPI (Invitrogen). The cellular localization of the mTH construct with or without the co-expression of Vpu was analyzed using the Perkin Elmer Ultraview ERS laser spinning disk confocal imaging system at 100× magnification (PerkinElmer, Waltham, Mass.). Images were acquired using this microscope and processed using Volocity software (Improvision, PerkinElmer) and Adobe Photoshop Creative Suite 2. The HaloTag™ reporter used is only bound by the HaloTag™ ligand when it is expressed on a cell surface of non-permeabilized cells.

As can be seen in FIG. 8, Panel A, expression of the mTH construct resulted in localization of the construct on the cell surface of the host cell. Panel B shows that co-expression with HIV-1 Vpu eliminates the cell-surface localization. The mTH construct is thus fully capable of recapitulating the activity of Tetherin with respect to an anti-Tetherin, and thus is a suitable construct for use in assaying for Tetherin inhibition by anti-Tetherins and for identification of modulators of that inhibition. The HaloTag™ construct used relied on a detection system that included reagents that are impermeable to cells. Additional experiments were performed to determine the cellular localization of the mTH construct when co-expressed with Vpu by permeabilizing the HeLa cells. Those experiments showed that the mTH construct was distributed about the cytoplasm of the cell (data not shown).

### Example 5

### Identification of Sequences Sufficient for Tetherin Activity Recapitulation

The 50 residue stalk of human Tetherin was shown above to be sufficient for cell-surface localization and anti-Tetherin inhibition. To further define the important regions of this 50 residue portion of Tetherin, the transmembrane region and/or the cytoplasmic tail was replaced by the transmembrane anchor sequence from human transferrin receptor type-1 protein, a protein known not to be inhibited by anti-Tetherins. The various constructs that were created are depicted in cartoon form in FIG. 9A. FIG. 9B shows the results of expression of these constructs in the presence or absence of HIV-1 Vpu.

More specifically, FIG. 9B shows that full-length human Tetherin functioned as expected, reducing release of Virus-Like Particles (VLPs) by about 95%. Further, co-expression of Tetherin and HIV-1 Vpu caused a significant increase in the release of VLPs. In contrast, replacement of the transmembrane domain and cytoplasmic tail with the corresponding sequences of human transferrin receptor eliminated the effect of Vpu on the anti-budding activity of Tetherin. Further, replacement of only the cytoplasmic tail region of human Tetherin did not have a significant effect on Vpu inhibition of the Tetherin. However, replacement of only the transmembrane domain of Tetherin with the transferrin receptor sequence essentially eliminated the Vpu inhibition activity. In the Figure, the solid arrows indicate Vpu activity, while the dashed arrows indicate lack of Vpu activity.

The experiments presented in FIG. 9B indicate that the transmembrane domain region of Tetherin is not only a region that is important in localization of Tetherin to the cell membrane, but is also a region that is required for the inhibitory activity of Vpu and other anti-Tetherins.

Previous reports have concluded that the N-terminal cytoplasmic tail of Tetherins is important for proper cellular localization of Tetherins (Neil et al., 2008). However, the data provided herein shows that the cytoplasmic tail is not a critical element for cellular localization and inhibition by certain anti-Tetherins (e.g., (HIV-1 Vpu, Ebola GP and HIV-2 Env). Specifically, data provided herein show that the cytoplasmic tail can be replaced with sequences from other proteins showing similar functions (i.e., type II transmembrane proteins). The cytoplasmic tail is thus not a critical element for recapitulation of Tetherin anti-viral budding activity, for proper cellular localization, or for inhibition by certain anti-Tetherins. However, KSHV K5 is not able to inhibit Tetherin derivatives substituted for the cytoplasmic tail of transferrin receptor, indicating that K5 targets Tetherin in an interaction that requires the cytoplasmic tail fo Tetherin.

It will be apparent to those skilled in the art that various modifications and variations can be made in the practice of the present invention without departing from the scope or spirit of the invention. Other embodiments of the invention will be apparent to those skilled in the art from consideration of the specification and practice of the invention. It is intended that the specification and examples be considered as exemplary only, with a true scope and spirit of the invention being indicated by the following claims.

1. An engineered protein comprising
a first amino acid sequence sufficient for localizing the protein to a surface of a cell and sufficient for inhibition of cell surface localization by an anti-Tetherin, wherein the sequence is not the full-length sequence of a naturally occurring Tetherin; and
a second amino acid sequence that produces or participates in the production of a detectable signal.

2. The protein of claim 1, wherein the first amino acid sequence includes the transmembrane domain region of a Tetherin protein.

3. The protein of claim 1, wherein the first amino acid sequence includes the transmembrane domain region of a Tetherin protein and the cytoplasmic tail region of a Tetherin protein.

4. The protein of claim 1, wherein the second amino acid sequence includes an intrinsically fluorescent protein.

5. The protein of claim 1, wherein the second amino acid sequence includes an enzyme.

6. A nucleic acid encoding a fusion protein, wherein the nucleic acid comprises
a first coding sequence for an amino acid sequence that is sufficient for localizing the protein to a surface of a cell and sufficient for inhibition of cell surface localization by an anti-Tetherin; and
a second coding sequence for a polyamino acid that produces or participates in the production of a detectable signal.

7. The nucleic acid of claim 6, wherein the first coding sequence includes the coding sequence for a transmembrane domain region of a Tetherin protein.

8. The nucleic acid of claim 6, wherein the first coding sequence includes the coding sequence for a transmembrane domain region of a Tetherin protein and the cytoplasmic tail region of a Tetherin protein.

9. The nucleic acid of claim 6, further comprising a coding sequence for an anti-Tetherin.

10. A recombinant cell comprising the nucleic acid of claim 9.

11. A recombinant cell comprising the nucleic acid of claim 6.

12. A recombinant cell comprising the protein of claim 1.

13. A method of identifying one or more bioactive agents capable of interfering with the ability of an anti-Tetherin to inhibit a Tetherin, said method comprising:
expressing a Tetherin reporter construct in a cell, wherein the Tetherin reporter construct comprises a first amino acid sequence sufficient for localizing the protein to a surface of a cell and sufficient for inhibition of cell surface localization by an anti-Tetherin, and a second amino acid sequence that produces or participates in the production of a detectable signal;
expressing an anti-Tetherin in the cell;
exposing the Tetherin reporter construct and anti-Tetherin, within the cell, to one or more substances; and
determining the cellular location of the Tetherin reporter construct,
wherein substantial localization of the Tetherin reporter construct at the cell surface indicates that the substance(s) include a bioactive agent capable of interfering with the ability of an anti-Tetherin to inhibit a Tetherin, whereas substantial localization of the Tetherin reporter construct in the cell cytoplasm indicates that the substance(s) do not include a bioactive agent capable of interfering with the ability of an anti-Tetherin to inhibit a Tetherin.

14. The method of claim 13, wherein the step of determining the cellular location includes detecting an optical signal produced by the Tetherin reporter construct.

15. The method of claim 13, wherein the Tetherin reporter construct and anti-Tetherin are expressed from the same nucleic acid molecule.

16. The method of claim 13, wherein the step of determining the cellular location includes confocal microscopy.

17. The method of claim 13, which is a method of High-Throughput Screening (HTS).


SEQUENCE LISTING (13 sequences)

SEQ ID NO: 1 - protein, 180 aa, Homo sapiens
MASTSYDYCRVPMEDGDKRCKLLLGIGILVLLIIVILGVPLIIFTIKANSEACRDGLRAVMECRNVTHLLQQELTEAQKGFQDVEAQAATCNHTVMALMASLDAEKAQGQKKVEELEGEITTLNHKLQDASAEVERLRRENQVLSVRIADKKYYPSSQDSSSAAAPQLLIVLLGLSALLQ

SEQ ID NO: 2 - protein, 180 aa, Chlorocebus aethiops; identical to SEQ ID NO: 1

SEQ ID NO: 3 - protein, 182 aa, Macaque fasicularis
MAPILYDYCKMPMDDIWKEDGDKRCKPVIGILGLLVIVLLGVLLIFFTIKANSEACQDGLRAVMECRNVTYLLQQELAEAQRGFRDAEAQAVTCNQTVMALMASLDAEKAQGRKKVEELEGEITTLNHKLQDASAEVERLRRENHVLNARIADTDSASSQDSSCAAEPPLLILLLGLSALLL

SEQ ID NO: 4 - protein, 181 aa, Macaque mulatta
MAPILYDYCKMPMDDIWKEDGDKRCKLVIGILGLLVIVLLGVLLIFFTIKANSEACQDGLRAVMECRNVTYLLQQELAEAQRGFRDAEAQAVTCNQTVMALMASLDAEKAQGRKKVEELEGEITTLNHKLQDSAEVERLRRENHVLNARIADTDSGSSQDSSCAAEPPVLILLLGLSALLL

SEQ ID NO: 5 - protein, 181 aa, Macaque nemistrina
MAPILYDYCKMPMDDIWKEDGDKRCKLVVGILGLLVIVLLGVLLIFFTIKANSEACQDGLRAVMECRNVTYLLQQELAEAQRGFRDAEAQAVTCNQTVMALMASLDAEKAQGRKKVEELEGEITTLNDKLQDSAEVERLRRENHVLNARIADTDSASSQDSSCAAEPPLLILLLGLSALLL

SEQ ID NO: 6 - protein, 184 aa, Pan troglodytes
MASTLYDYCRVPMDDIWKKDGDKRCKLLLGIGILMLLIIVILGVPLIIFTIKANSEACRDGLRAVMECRNVTHLLQQELTEAQKGFQDVEAQAATCNHTVMALMASLDAEKAQGQKKVEELEEEITTLNHKLQDSAEVERLRRENQVLSVRIADKKYYSSSQDSSSAAAPQLLIVLLGLSALLQ

SEQ ID NO: 7 - DNA, 983 bp, Homo sapiens
gtggaattcatggcatctacttcgtatgactattgcagagtgcccatggaagacggggataagcgctgtaagcttctgctggggataggaattctggtgctcctgatcatcgtgattctgggggtgcccttgattatcttcaccatcaaggccaacagcgaggcctgccgggacggccttcgggcagtgatggagtgtcgcaatgtcacccatctcctgcaacaagagctgaccgaggcccagaagggctttcaggatgtggaggcccaggccgccacctgcaaccacactgtgatggccctaatggcttccctggatgcagagaaggcccaaggacaaaagaaagtggaggagcttgagggagagatcactacattaaaccataagcttcaggacgcgtctgcagaggtggagcgactgagaagagaaaaccaggtcttaagcgtgagaatcgcggacaagaagtactaccccagctcccaggactccagctccgctgcggcgccccagctgctgattgtgctgctgggcctcagcgctctgctgcagtgagatcccaggaagctggcacatcttggaaggtccgtcctgctcggcttttcgcttgaacattcccttgatctcatcagttctgagcgggtcatggggcaacacggttagcggggagagcacggggtagccggagaagggcctctggagcaggtctggaggggccatggggcagtcctgggtgtggggacacagtcgggttgacccagggctgtctccctccagagcctccctccggacaatgagtcccccctcttgtctcccaccctgagattgggcatggggtgcggtgtggggggcatgtgctgcctgttgttatgggttttttttgcggggggggttgcttttttctggggtctttgagctccaaaaaataaacacttcctttgagggagagcaaaaaaaaaaaaaaaaaaaaaaaaaaaaaaaaaaaa

SEQ ID NO: 8 - DNA, 66 bp, Artificial sequence
  Consensus nucleic acid sequence for transmembrane domain of Tetherins.
nnnnnnnnnnnnnnnggaattctgnnnctcctgnnnatcgtgnnnctgggggtgnnnttgattnnn

SEQ ID NO: 9 - DNA, 144 bp, Artificial sequence
  Consensus nucleic acid sequence for Tetherin transmembrane domain and cytoplasmic tail
atggcannnnnnnnntatgactatnnnnnnnnncccatgnnnnnnnnnnnnnnnnnngacnnngataagnnntgtaagnnnnnnnnnnnnnnnggaattctgnnnctcctgnnnatcgtgnnnctgggggtgnnnttgattnnn

SEQ ID NO: 10 - protein, 290 aa, Artificial sequence
  Amino acid sequence of mTG reporter construct
MASTSYDYCRVPMEDGDKRCKLLLGIGILVLLIIVILGVPLIIFTIKANSGGVSKGAELFTGIVPILIELNGDVNGHKFSVSGEGEGDATYGKLTLKFICTTGKLPVPWPTLVTTLSYGVQCFSRYPDHMKQHDFFKSAMPEGYIQERTIFFEDDGNYKSRAEVKFEGDTLVNRIELTGTDFKEDGNILGNKMEYNYNAHNVYIMTDKAKNGIKVNFKIRHNIEDGSVQLADHYQQNTPIGDGPVLLPDNHYLSTQSALSKDPNEKRDHMIYFGFVTAAAITHGMDELYK

SEQ ID NO: 11 - protein, 50 aa, Artificial sequence
  Amino acid sequence of human Tetherin “stalk” peptide
MASTSYDYCRVPMEDGDKRCKLLLGIGILVLLIIVILGVPLIIFTIKANS

SEQ ID NO: 12 - protein, 21 aa, Artificial sequence
  Consensus Tetherin transmembrane sequence
XXXXXGILXLLXIVXLGVXLI

SEQ ID NO: 13 - protein, 55 aa, Artificial sequence
  Consensus amino acid sequence of Tetherin “stalk” peptide
MAXXXYDYXXXPMXXXXXXDXDKXCKXXXXXGILXLLXIVXLGVXLIXFXIKANS